(19) United States
(12) Patent Application Publication
(10) Pub. No.: US 2005/0197609 A1
(43) Pub. Date: Sep. 8, 2005

(54) Wrist brace having twistable elements and method of using the same

(75) Inventor: Jessica A. Mills, Mason, OH (US)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf, Inc.

(21) Appl. No.: 10/793,394

(22) Filed: Mar. 4, 2004

(51) Int. Cl. A61F 5/00; A61F 13/00

(52) U.S. Cl. 602/21; 602/64

(57) ABSTRACT

A reversible wrist brace includes a sheet of flexible material that overlies and supports a wearer's wrist. The sheet has at least one connecting element connected to a lateral edge at one end and to at least one fastener at its opposite end. The connecting element also includes a twistable element positioned between the ends of the connecting element such that the fastener may twist relative to the flexible sheet. The fastener and connecting element enable a wearer to switch the wrist brace between right and left hands by twisting the twistable element to orient a fastening surface on the fastener so that it is coincident with an outer surface of the sheet of material regardless of whether the wrist brace is worn on either the right or left hand. The brace may include a splint that can be inserted within a pocket located on the sheet to further immobilize the wrist.

### BACKGROUND OF THE INVENTION

1. Field of the Invention

The present invention is related to the field of orthopedic braces, and more particularly, to braces for supporting a wrist joint of a wearer.

2. Description of Related Art

Wrist injuries are relatively common orthopedic injuries that occur in a range of environments, such as a home, office or outdoor environment, and can have varying levels of severity. These levels of severity are routinely grouped into two categories, chronic and acute. Acute injuries occur most often due to sudden impacts that involve the large forces caused by an accident or from a collision during athletic activities. Such traumatic injuries to the wrist typically involve broken bones or sprains. Broken bones are usually treated using complete immobilization, while sprains often only require moderate immobilization for a period of weeks depending on the grade of the sprain. Establishing immobilization early after a sprain ensures that the injury does not evolve into a chronic injury.

Repetitive stresses to the wrist joint are believed to be the most common cause of chronic injuries. Repetitive stresses often occur in an office environment, e.g., typing, or during labor activities, e.g., assembly line work. Given the range of environments and types of injuries that can occur to the wrist, it is advantageous to have wrist braces that provide a range of flexibility and support for the wearer. In addition, due to the reoccurring, extended duration of chronic wrist injuries, a comfortable wrist brace is also desirable.

U.S. Pat. No. 6,561,994 to Mills et al. (“Mills”) discloses a wrist brace 10 having both a stretchable portion 12 and a non-stretchable portion 13, as shown in FIG. 1 of Mills. Mills also discloses fasteners having hook or loop material 19, 20, and 21 as well as a longitudinal pocket 22 having a portion of complimentary hook or loop material 22. The wrist brace also includes a splint 30 that may be inserted within the longitudinal pocket 22. The Mills patent is also described as being reversible from either the right or the left hand, at column 2, lines 36-37. A reversible brace enables a wearer to use the same brace for injuries of either the right or left wrists.

The material disclosed in the Mills patent is fabricated of a portion of stretchable material 12 that is capable of stretching to different wearer's wrist sizes when worn. With the fasteners being attached to the elastic material, the wearer can adjust the wrist brace to the desired tension and support depending on the amount of immobilization that is required for a wearer's particular injury. Typically, more immobilization would be required for acute injuries, while less immobilization would be required for chronic injuries so that a wearer can still continue with normal activities while the wrist brace is being worn.

As shown in FIGS. 5-8, the Mills patent also discloses a splint 30 that is formed with a curvature 32 to conform to the wearer's wrist and palm, whereby the splint is inserted into the pocket 22 of the wrist brace, and the wrist support is wrapped around the wearer's wrist and hand and secured with fastening straps 18, 19, and 20. The splint is generally inflexible and provides more immobilization to the wearer's wrist when inserted within the pocket. Thus, the splint would most often be used with acute type injuries, or those chronic injuries where more immobilization is desired.

The various material portions, the fasteners, and the splint provide the versatility to adjust the amount of immobilization depending on the type of injury involved. Mills also discloses a brace that is easy to use given the configuration of the fasteners and flexible material. Despite these improvements, additional innovations in wrist braces to promote better treatment of wrist injuries are also desired.

It would be advantageous to provide a wrist brace that can be adjusted to provide support for a range of injuries, both chronic and acute. In addition, it would be advantageous to have a wrist brace that is easy to use. Finally, it would also be desirable to have a wrist brace that can be used on either a right or left wrist of a wearer.

### BRIEF SUMMARY OF THE INVENTION

The above-listed objectives, and other advantages, are achieved by providing a wrist brace of the present invention for supporting a wrist of the wearer. The reversible wrist brace includes a sheet of flexible material that overlies and supports a wearer's wrist. The wrist brace has at least one connecting element connected to a lateral edge at one end and to at least one fastener at its opposite end. The connecting element also includes a twistable element that is positioned between the ends of the connecting element such that the fastener may twist relative to the sheet of material. The fastener and connecting element enable a wearer to switch the wrist brace between right and left hands by twisting the twistable element to orient a fastening surface on the fastener so that it is coincident with, and attachable to, an outer surface of the sheet of material. The brace may also include a splint that can be inserted within a pocket located on the sheet of material to further immobilize the wrist. Several of the fasteners and connecting elements may be employed to vary the amount of support provided by the wrist brace.

In one embodiment, the present invention includes a reversible wrist brace for alternatively supporting both a right and left wrist of a wearer. A sheet of flexible material of the reversible wrist brace has opposed first and second surfaces that are configured to overlie the right and left wrists, respectively. Also, the sheet of material includes a distal edge that is configured to extend at least partially around the wearer's hand, a proximal edge configured to extend at least partially around the wearer's forearm, and a pair of opposing lateral edges capable of at least partially overlapping each other to extend around the wearer's wrist.

The wrist brace also includes at least one fastener having a fastening surface and a non-fastening surface, wherein the fastening surface is configured to attach to a portion of the first or second surfaces of the sheet. In addition, the wrist brace includes at least one connecting element having a first end connected to the sheet of material and a second end connected to the fastener. The connecting element includes a twistable element positioned between the ends of the connecting element. Advantageously, the twistable element is configured to allow sufficient rotation between the ends to allow reorientation of the fastening surface. Thus, the fastening surface may be oriented between being attached to the first or second surfaces to secure the sheet about the wearer's left and right wrists, respectively. In variations of this embodiment, the twistable element may twist up to 180° or up to 360° or even beyond.

In another embodiment, the twistable element includes a first member attached to the first end and a second member attached to the second end. One of the members defines an opening, and the other one of the members is rotatably retained within the opening. In one version, the second member is rotatable through 360° with respect to the first member when retained within openings defined in the first member. Optionally, the twistable element may further include a third member extending between the first member and the first end. The third member is attached to the first end, and the first member is rotatable with respect to the third member about an axis different than an axis of rotation of the first and second members. For example, the third member can have a U-shape with a pair of arms, wherein the first member is rotatably supported between the arms. The wrist brace may also include a strap extending through the U-shape and between the U-shape and the first member, wherein the strap is attached to the sheet at one end.

In yet another embodiment, the twistable element may include a flexible tether. The flexible tether may be biased to an untwisted position, wherein the fastening surface is coincident with one of the first and second surfaces of the flexible sheet of material. Such biasing may be accomplished by the tether having a cross-section with a primary axis aligned with the first surface or the second surface of the sheet of material.

The present invention has many advantages. The construction of the twistable element allows the wearer to quickly and easily rotate each of the fasteners when switching between right and left-handed wrist brace configurations. Reversal of the fastening surface when the fastening surface comprises at most one side of each of the fasteners ensures that the fastening surface is not exposed. This prevents abrasion of the wearer's skin by the fastening surface or snagging of the fastening surface on the wearer's clothing. Also, hiding or concealing the fastening surface can improve the aesthetic appearance of the wrist brace. The use of multiple twistable elements in conjunction with multiple fasteners (e.g., three twistable elements) at different positions along a lateral edge of the sheet of flexible material allows adjustment of the amount of support provided by each of the fasteners at different locations.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 is a perspective view of a wrist brace of one embodiment of the present invention;

FIG. 2 is a plan view of a first surface of the wrist brace shown in FIG. 1;

FIG. 3 is a plan view of a second opposite surface of the wrist brace shown in FIG. 1;

FIG. 4 is an enlarged view of a twistable element positioned between a fastener and the sheet of material of the wrist brace of FIG. 1;

FIG. 5 is another perspective view of the wrist brace of FIG. 1;

FIG. 6 is another perspective view the wrist brace of FIG. 1;

FIG. 7 is a plan view of a splint of the wrist brace of FIG. 1;

FIG. 8 is a side elevation view of the splint of FIG. 7;

FIG. 9 is a plan view of a first surface of a wrist brace of another embodiment of the present invention;

FIG. 10 is a plan view of a second opposite surface of the wrist brace shown in FIG. 9;

FIG. 11 is an enlarged view of a twistable element positioned between a fastener and the sheet of material of the wrist brace of FIG. 9;

FIG. 12 is another perspective view of the wrist brace of FIG. 9;

FIG. 13 is another perspective view the wrist brace of FIG. 9; and

FIG. 14 is an enlarged view of a twistable element of another embodiment of the present invention.

### DETAILED DESCRIPTION OF THE INVENTION

The present invention now will be described more fully hereinafter with reference to the accompanying drawings, in which some, but not all embodiments of the invention are shown. Indeed, the invention may be embodied in many different forms and should not be construed as limited to the embodiments set forth herein; rather, these embodiments are provided so that this disclosure will satisfy applicable legal requirements. Like numbers refer to like elements throughout.

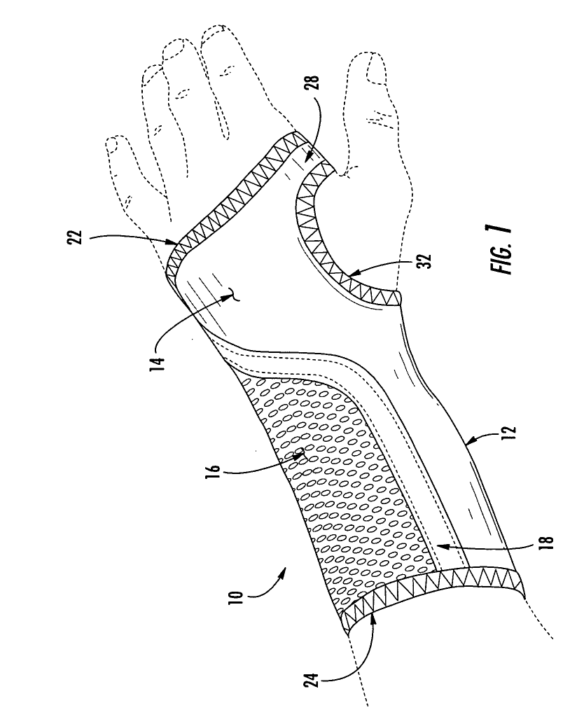

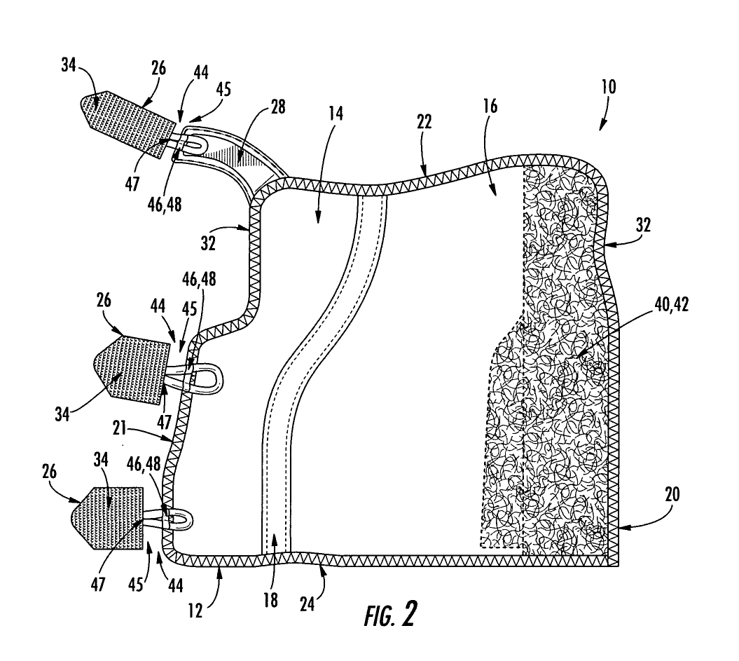

One embodiment of a wrist brace 10 of the present invention is shown in FIGS. 1 and 2. The wrist brace 10 includes a sheet 12 of material defined by edges that can extend around the wrist of a wearer. Closure of the sheet of material is secured using a plurality of fasteners 26. Advantageously, the wrist brace 10 is reversible and can be used on either the right or left wrists of the wearer because of the reversible orientation of the sheet of material 12 and the fasteners 26, as will be described in more detail below.

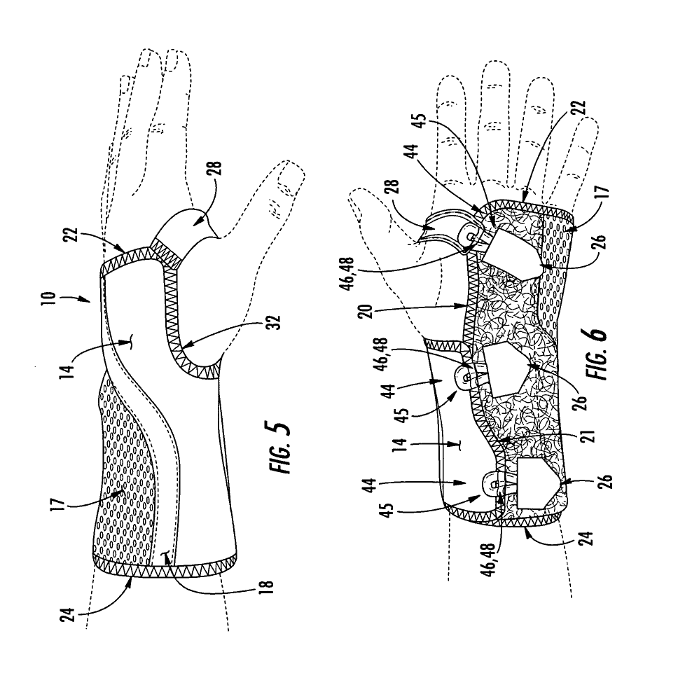

Preferably, the sheet of material 12 has a pair of lateral edges 20 and 21, a distal edge 22 and a proximal edge 24. In this manner, when the wrist brace 10 is applied to a wearer's wrist the distal edge 22 extends around the wearer's hand, the proximal edge 24 extends around the wearer's forearm, and the opposing lateral edges 20, 21 overlap each other so as to extend around the wearer's wrist, as is illustrated in FIGS. 1, 5, and 6.

Although the illustrated embodiment shows the sheet of material 12 as having a somewhat rectangular shape with four relatively discrete edges, the sheet of material could have a range of shapes and edge constructions, as long as it is capable of at least partially extending around portions of the hand, arm and wrist. For instance, the sheet of flexible material 12 here could be a circular sheet capable of having only one discrete, circular edge wherein arc-portions of the circular edge extend around the wearer's hand, forearm and wrist and serve as the distal edge 22, the proximal edge 24 and the opposing lateral edges 20, 21.

Optionally, opposite portions of the lateral edges 20 and 21 of the sheet of material 12 (near the distal edge 22) curve inwardly so as to form a pair of inwardly curved edges 32, as shown in FIG. 2. The curved edges 32 form an opening around the wearer's thumb when the remaining portions of the lateral edges 20, 21 are brought together in an adjacent/overlapping arrangement.

In another aspect, the sheet of material 12 may include multiple panels or portions. For instance, in the illustrated embodiment the sheet of material includes a stretchable material portion 14 and a non-stretchable material portion 16, as shown in FIG. 2. The stretchable material portion 14 and non-stretchable material portion 16 have inner edges that are joined along a junction 18 having a spiraled shape. In particular, the spiraled junction is generally closer to the lateral edge 20 at the distal edge 22 of the sheet of material and extends further away from the lateral edge 20 (i.e., towards the opposite lateral edge 21) as it extends toward the proximal edge 24 of the sheet of material. Therefore, in one embodiment, the non-stretchable material portion 16 is wider at the proximal edge 24 than at the distal edge 22.

The non-stretchable material portion 16 of the wrist brace 10 may be constructed of any fabric that is non-stretchable or substantially non-stretchable, but is preferably made of non-latex material to avoid allergic reactions. Generally, the non-stretchable material portion 16 should be comfortable and breathable so as to wick away moisture from the wearer's hand. As another option, holes 17 may be provided in the non-stretchable material portion 16 to provide additional ventilation and make the material portion even more breathable.

Examples of non-stretchable materials that could be used include material marketed by GEHRING TEXTILES, INC. as SPACER FABRICS, and those marketed by GUILFORD HILLS, INC. as COOL FLEX fabric. Another example of a material or fabric that can be used as the non-stretchable material portion 16 is the warp knitted textile spacer fabric disclosed in U.S. Pat. No. 5,385,036. It should be noted that instead of just a portion, the sheet of material 12 may be entirely formed of a non-stretchable material, especially in circumstances where the wrist brace 10 is custom-fit to an individual wearer.

The stretchable material portion 14 of the wrist brace 10 may be constructed of any variety of flexible and elastic materials so as to promote extension of the fasteners 26 around the hand and wrist to achieve the desired fit. For instance, the stretchable material portion 14 may be constructed of a fabric that is elastic in one or more directions. Preferably, however, the stretchable material portion 14 is elastic in at least the lateral direction (i.e., between the lateral edges 20, 21) to promote easy extension of the sheet of material 12 about the wrist. As another option, the stretchable material portion 14 may be elastic in both the lateral and longitudinal directions (i.e., between the distal edge 22 and the proximal edge 24). For further comfort, the stretchable material portion 14 may even be elastic in three directions.

Examples of materials well-suited for use in the stretchable material portion 14 are woven, non-woven or knit elastics, neoprene blends, foams, or laminates of the same. Latex materials such as neoprene may be used, but these are preferably avoided to reduce the chance of allergic reactions.

It should be noted that although the illustrated embodiments have stretchable and non-stretchable portions, the entire sheet of material 12 may be formed of a stretchable material, especially in circumstances where fitting of a larger range of hand, wrist and arm sizes is desired. In addition, it should be noted that the sheet of flexible material 12 could include any number of elastic or inelastic material portions as long as it has sufficient overall flexibility to extend around, and provide support for, the wearer's wrist.

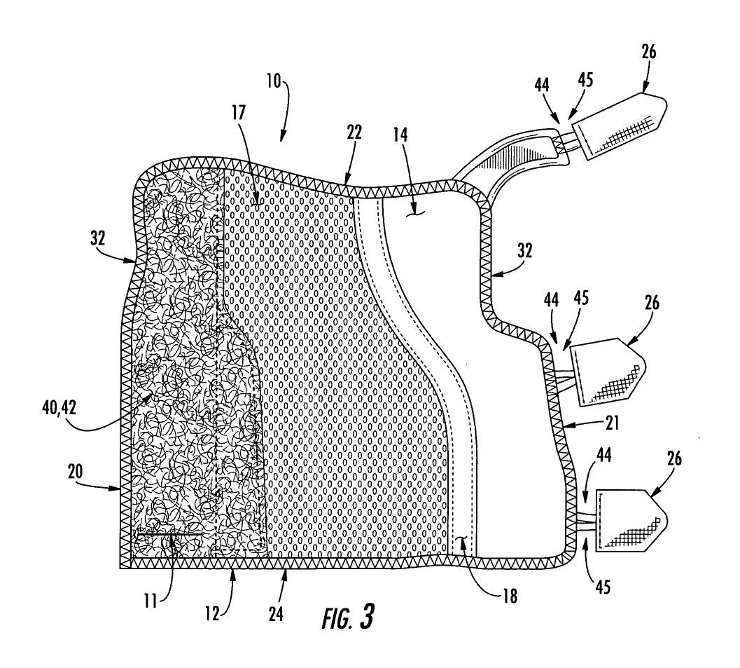

As another option, the sheet of material 12 may include a pocket 42 for accommodating an elongated splint 54, as shown in FIGS. 3, 7, and 8. The pocket 42 can be located on either side of the sheet of material 12 and is preferably positioned along a lateral edge of the sheet of material opposite the lateral edge 21 and the fasteners 26. In the illustrated embodiments, the pocket 42 is sufficiently long in the proximal-distal direction to extend substantially from the proximal edge 24 to the distal edge 22 of the sheet 12 of flexible material. This allows the pocket 42 to accommodate a relatively long elongated splint 54. It should be noted that although the illustrated embodiments are shown as having a pocket 42 and splint 54, the pocket and splint may not even be necessary for wrist brace embodiments, especially where lesser amounts of immobilization are preferred (e.g., for chronic injuries).

Preferably, an opening is defined in the material of the pocket 42 so as to allow insertion, removal, and reversal of the orientation of the elongated splint 54. For instance, in the illustrated embodiment a slit 11 is defined in the pocket 42 adjacent the proximal edge 24, as shown in FIG. 3. Of course the slit or other opening could be defined elsewhere in the pocket 42, but its position in the illustrated embodiment facilitates easy insertion of one end of the elongated splint 54. The slit 11 could also be defined adjacent the distal edge 22 and still advantageously allow similar end-wise insertion of the splint 54.

The pocket 42 is preferably constructed of a non-stretchable material that also acts as a complementary fastening surface 40 (e.g., a loop-bearing material) for the fasteners 26. However, the pocket 42 could also be an elastic or stretchable material that does not act as a fastening surface 40. In such a case, a separate complementary fastening surface 40 may simply be attached to the exterior surface of the pocket 42. For an ambidextrous wrist brace, both sides of the pocket 42 can include the complementary fastening surface 40 so that fastening surfaces on the fasteners 26 may attach to either side of the sheet.

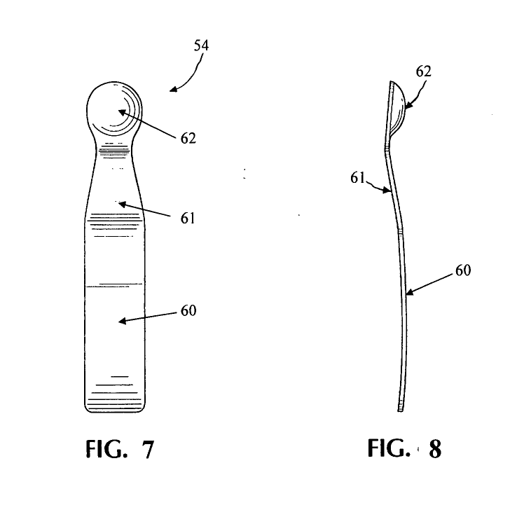

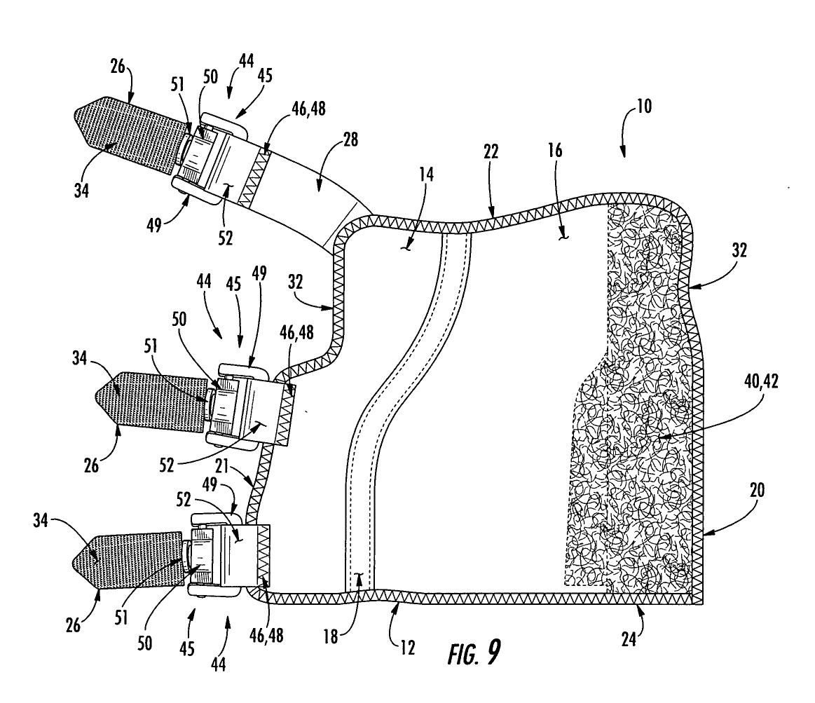

Referring again to FIGS. 7 and 8, the splint 54 for insertion into the pocket 42 has an elongate shape that includes a planar portion 60, an arc portion 61 and a crown portion 62. Preferably, the splint 54 is shaped from an elongate, roughly rectangular sheet of rigid material, e.g., aluminum sheet material shaped using a stamping process. The planar portion 60 comprises about half of the length of the splint 54 and generally retains the original, unstamped shape of the sheet of material. The arc portion 61 extends away from the plane of the planar portion in an arc between the planar portion 60 and the crown portion 62. The crown portion 62 has a slightly convex curvature and extends away from the arc portion 61 at an angle more oriented in parallel with the planar portion 60.

The longitudinal and lateral dimensions of the splint 54 are such that the splint extends substantially from the proximal edge 24 to the distal edge 20 along the length of the pocket 42. When the splint 54 is positioned within the pocket 42 the crown and arc portions 62, 61 align with the wearer's palm and wrist, respectively, to maintain the wearer's hand in extension relative to the wrist. Also when in this position, as shown in FIG. 5, the planar portion 60 extends along the wearer's forearm. Such a position is typically considered anatomically neutral so as to reduce strain on the wrist.

It should be noted that if employed, the splint 54 could have any number of shapes, sizes and materials (steel, wood, plastic, etc.) so as to accommodate different wrist sizes, as well as to adjust the desired amount of immobilization. For instance, the splint could be planar along its length, as opposed to the shaped splint of the illustrated embodiments.

In the illustrated embodiment of FIGS. 2 and 3, the wrist brace 10 is shown as having three fasteners 26, where a first fastener is located near the distal edge 22, a second fastener is located near the proximal edge 24, and a third fastener is located between the first and second fasteners. However, the wrist brace 10 of the present invention could have one, two, or more than the three fasteners 26 of the illustrated embodiment, depending upon the size and coverage of the sheet of material 12, the size of the fasteners themselves, the desired tightness of the fit of the wrist brace, etc. For instance, only a single fastener 26 may be needed if the fastener is very wide, extending over the entire wrist.

As shown in FIGS. 2-3 and 9-10, the wrist brace 10 may also include a thumb tab 28 that is connected at one end to a corner of the sheet of material 12 between the distal edge 22 and the lateral edge 21. Connected to the thumb tab 28 is one of the fasteners 26. Advantageously, the thumb tab 28 is sufficiently long to extend between the thumb and index finger of the wearer so that attachment of the fastener can encircle the wearer's thumb, as shown in FIG. 1. The thumb tab 28 may be constructed of a range of flexible materials, such as the stretchable and non-stretchable materials described above for the sheet of material 12. As another option, the thumb tab may be integrally constructed as part of the sheet of material 12, such as part of the stretchable material portion 14.

Fastening by the fasteners 26 is preferably accomplished via some type of fastening surface 34 on one surface of each of the fasteners. In the illustrated embodiments, the fastening surface 34 is a hook material that can be connected to the complementary loop material on the pocket 42, such as the hook and loop material sold under the trademark VELCRO®. However, the term “fastening surface” as used herein denotes any type of chemical, mechanical or other fastener that allows connection of two separate components, such as snaps, hook and loop connectors, adhesives, buckles, etc. Thus, in one option, the pocket 42 could not include a complementary fastening surface 40 at all when the fasteners 26 attach via another mechanism than hook material, such as an adhesive. Notably, the fastening surface 34 (hooks) located on the outer surface of the fasteners 26 and the fastening surface 40 (loops) located on the sheet of material 12 mate to and attach with one another when brought into contact. These fastening surfaces 34, 40, therefore, are referred to herein as being complementary.

Regardless of the type of fastening surface 34 used for the fasteners 26 or the number of fasteners, each of the fasteners is connected to the sheet of flexible material 12 by a connecting element of the present invention that allows reorientation of the fasteners to promote reversibility of the wrist brace 10.

Each connecting element includes a first end connected to the sheet of material 12 and a second end connected to its respective one of the fasteners 26. Extending between these two ends is a twistable element such as a flexible tether, or a collection of multiple movable parts, that facilitate rotation of the fasteners 26 when switching between right and left-handed brace configurations, as will be described in greater detail hereinbelow.

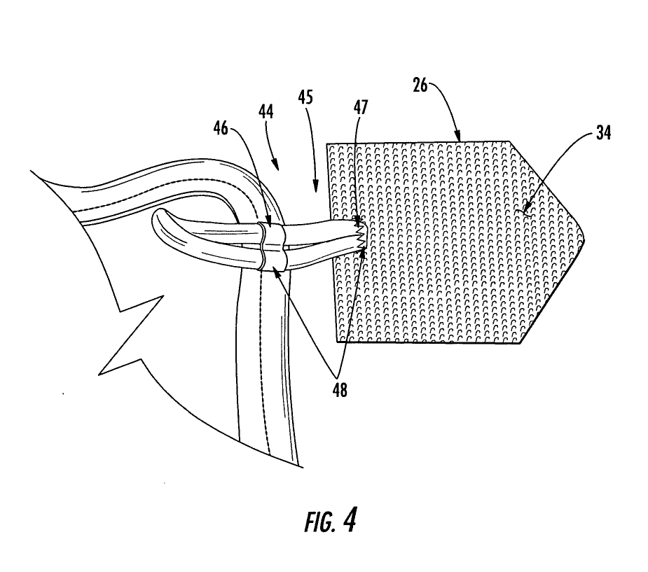

In the embodiment of the wrist brace 10 of the present invention illustrated in FIGS. 1-6, a connecting element includes a single, flexible cord 44 that has been folded over onto itself to form a tether. In particular, the flexible cord 44 includes a first end 46 that is attached to the sheet of material 12 (proximal to the lateral edge 21) with stitching 48, as is shown best in FIG. 4. Similarly, the flexible cord 44 is attached with stitching 48 at its opposite, second end 47 to its respective one of the fasteners 26. An intervening length of the flexible cord 44 between the ends 46, 47 serves as a twistable element 45 of the flexible cord 44 so as to allow reorientation of the fastener attached thereto.

It should be noted that the flexible cord 44 can have varying lengths, diameters, or thicknesses depending upon a number of factors, such as the size of the wearer's wrist, hand and forearm, the number of fasteners, the dimensions of the sheet of material 12. Preferably, however, the flexible cord 44 is approximately two inches in length which, when folded over, has a length of about one inch. Once connected to the sheet of material 12, the twistable element 45 of the flexible cord 44 (between the ends 46, 47 as defined by the stitching 48) is reduced to about one-half of an inch.

In cross-section, the flexible cord 44 preferably has a non-circular shape with a primary axis defining a maximum width of the cross-section. A cross-section of single strand of the flexible cord 44 has a rectangular shape with a width of about one-eighth of an inch and a thickness of about one-sixteenth of an inch. When doubled over and attached using the stitching 48, the widths are in a side-by-side, adjacent relationship to form a primary axis of one-quarter of an inch and a thickness that is still one-sixteenth of an inch (a ratio of 4 to 1). Because of this, each of the fasteners 26 is biased towards an untwisted position in which the fastening surface 34 is oriented coincident with one surface of the sheet of material 12. Restated, if the twistable element 45 of the flexible cord 44 is twisted and subsequently released, the twistable element acts to untwist itself to its original orientation.

Although the flexible cord 44 has a generally rectangular cross-section, other cross-sections with and without primary axes may be used, such as circular cross-sections, square cross-sections or elliptical cross-sections. It should be noted that most materials will be biased to an untwisted position by their inherent elasticity even if they have a circular cross-section, but having a primary axis aligned with the sheet of material 12 facilitates untwisting. Also, it should be noted that there is somewhat of a tradeoff in that the greater the bias, the more resistance there will be to changing the orientation of the fasteners 26. Additionally, there may be some overall loss of the flexibility of the twistable element when its bias to an untwisted position is increased.

The twistable element 45 of the flexible cord 44 is preferably flexible enough for about 180 degrees of rotation or more so as to facilitate a full reversal of orientation of the fastening surface 34 to be coincident with the opposite surfaces of the sheet of material 12. The twistable element 45 of the flexible cord 44 could also twist less than 180 degrees in instances where the sheet of material 12 or the fasteners 26 are sufficiently flexible to allow attachment of the fastening surface 34, or where the fastening surface has an initial orientation that is less than 180 degrees from being able to attach to the sheet of material.

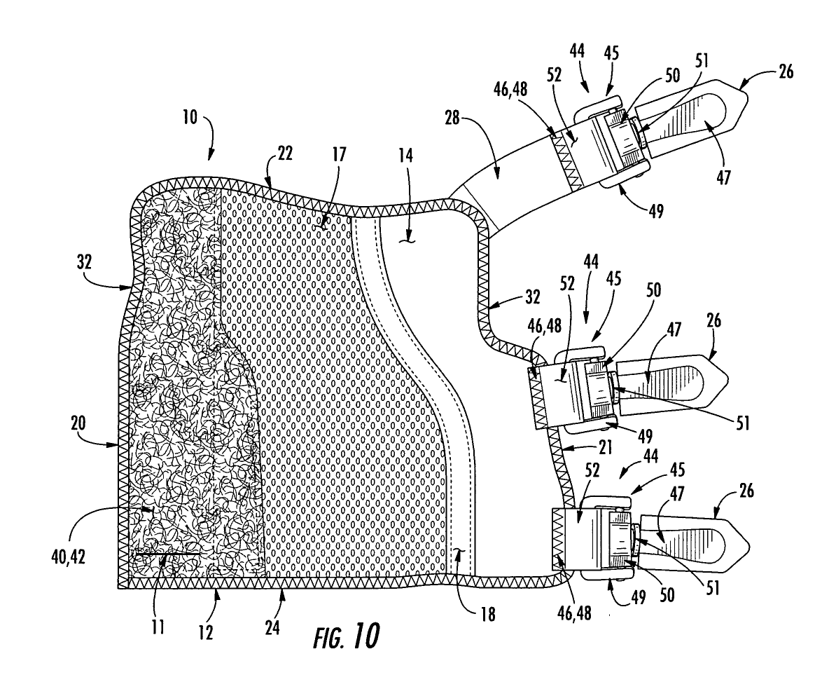

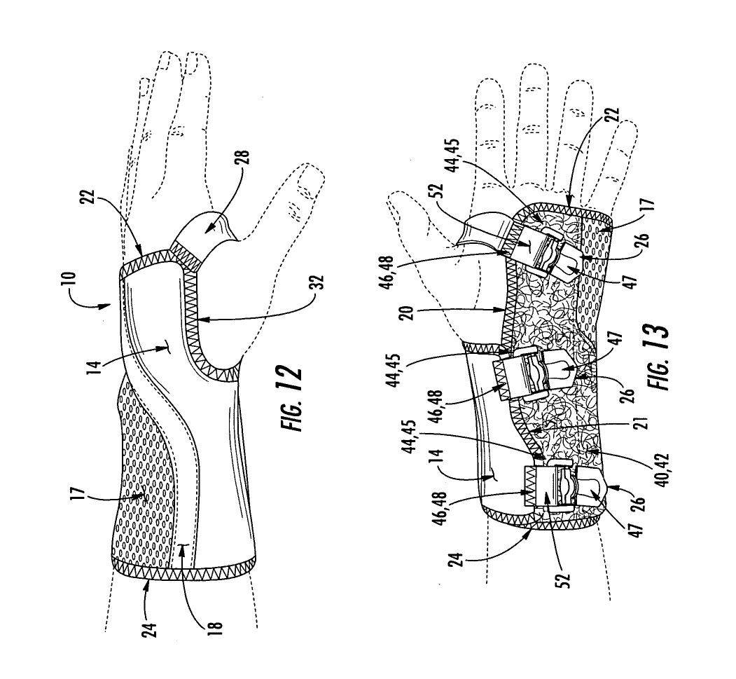

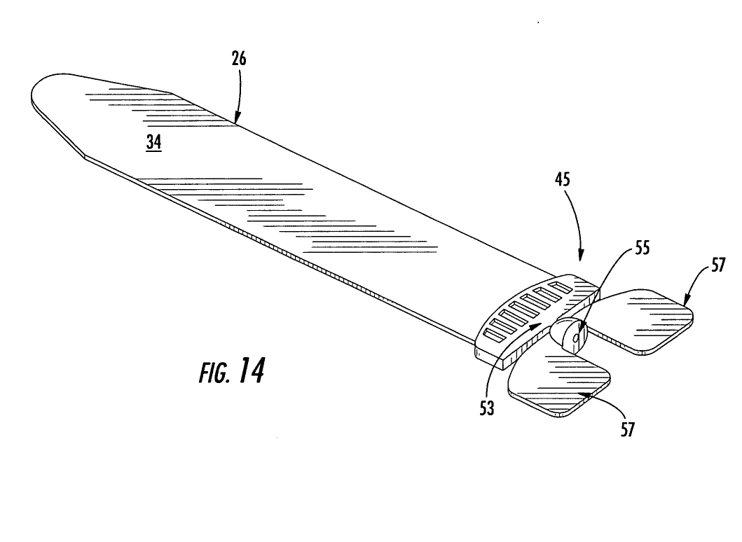

In another embodiment of the wrist brace 10 of the present invention illustrated in FIGS. 9-13, the connecting element includes a connecting assembly 44 of parts that are rotatable and/or translatable with respect to each other. In particular, the connecting assembly 44 has its first end 46 attached to the sheet of material 12 with stitching 48 and its second end 47 attached to one of the fasteners 26 with an adhesive. In this embodiment, the various moveable parts between the ends 46,47 serve as the twistable element 45 of the connecting assembly 44.

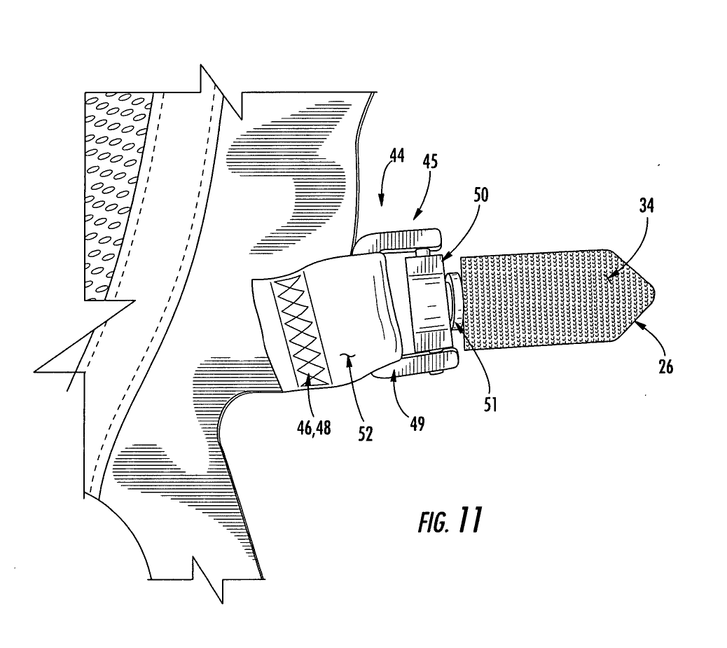

As shown in FIG. 11, the twistable element 45 of each connecting assembly 44 includes a U-shaped member 49 rotatably attached to the sheet of material 12 with a strap 52. The strap 52 is attached with the aforementioned stitching 48 on one surface of the sheet of material 12, loops over the U-shaped member 49, and attaches with stitching to the sheet of material on its opposite surface so as to retain the connecting assembly 44 at the lateral edge 21.

The twistable element 45 also includes a rotatable bar 50 that is rotatably coupled between the arms of the U-shaped member 49. In particular, the U-shaped member 49 defines a hole proximate to the free end of each of its arms that rotatably holds a respective end of the rotatable bar 50. The twistable element 45 further includes a swivel 51 rotatably mounted to the rotatable bar 50. In particular, the rotatable bar 50 defines a cylindrical hole approximately at its midpoint, wherein the swivel 51 is rotatably retained within the cylindrical hole.

Together, the movable components of the twistable element 45 of the connecting assembly 44 illustrated in FIGS. 9-13 are capable of rotating about multiple axes for placement and attachment of the fasteners 26 on either side of the sheet of material 12. In particular, the twistable elements 45 rotate freely about a first axis extending through the swivel 51, and also about a second axis extending through the rotatable bar 50, wherein the first and second axes are orthogonal (and are not collinear) with respect to each other. A third axis extends through the opening in the strap 52 such that the U-shaped member 49 may pivot within the strap 52. Because the size of the opening of the strap 52 is larger than the U-shaped member 49 it also allows some room for translation of the U-shaped member with respect to the strap, as well as rotation.

The range of rotation of the first axis is not limited as the swivel 51 rotates freely within the opening of the rotatable bar 50, while the second and third axes have limits to their rotation due to the size of the U-shaped member 49 and the limited flexibility of the strap 52. Combination of these axes of motion allows the fastening surface 34 of each of the fasteners 26 to be easily reoriented to mate with the complementary fastening surface 40 located on the pocket 42 on the sheet of material 12 when switching the wrist brace 10 between left and right-handed configurations.

The movable components of the twistable element 45 are preferably constructed of a relatively stiff material such as a rigid, molded plastic that allows relatively smooth rotation between the components. The strap 52 is preferably constructed of a relatively flexible sheet material, such as a leather or fabric material. However, it should be noted that the connecting elements described herein could be constructed of a range of suitable materials and still be within the purview of the present invention.

The movable components of the twistable element 45 may be varied in size and configuration depending upon such factors as the desired number of axes of rotation and the overall range of motion. For example, the free ends of the arms of the U-shaped member 49 are preferably separated by about one inch and are about ⅝ of an inch in length. The rotatable bar 50 is at least one inch in length so that the rotatable bar may extend within, and be retained by, the holes defined within the arms of the U-shaped member 49. However, the distance between the arms of the U-shaped member 49 could be increased to two inches, and the length of the rotatable bar 50 increased to two inches, and still facilitate reorientation of the fasteners 26.

It should also be noted that the twistable element 45 need not be limited to the particular configuration of the movable components illustrated in FIGS. 9-13. For example, FIG. 14 illustrates a twistable element 45 that includes a single swivel 53 that rotates within an opening defined in a flanged member 55 having a pair of flanges 57 which can be sewn into one of the lateral edges 20, 21 of the sheet of material 12. The axis of rotation of the swivel 53 allows a full 360 degrees of rotation for reorientation of the fastening surface 34.

It should also be noted that although the ends 46, 47 are described above as being attached using stitching and adhesive, they could be secured to the sheet of material 12 and the fasteners 26 in any manner sufficient to stay attached during tensioning of the connection element and attachment of the fasteners. For instance the ends 46, 47 could be attached using snaps, staples, buckles, hook and loop material, and the like, as long as the ends are relatively secure against separation when the fasteners 26 are being repositioned and secured to the outer surface of the sheet of material 12. As another option, the end 46 of the connecting element may be integrally constructed with the sheet of material 12, such as by being knit therewith. Similarly, the other end 47 may be integrally constructed with the fasteners 26, such as during a plastic extrusion process.

The term “twistable element” as used herein is not meant to be limiting and may include any flexible tether, two or more components of relatively movable parts or a combination of both. The twistable elements 45 could have many shapes and dimensions and still be within the purview of the present invention as long as the twistable elements can twist or rotate to change the relative orientation of the fastening surface 34 and the sheet of material 12. For instance, various types of tether could be a braided or unbraided cord, a woven or non-woven cord or strap, an elastic cord or strap, or the like with sufficient flexibility to allow rotation. The tether could have any number of dimensions and cross sections to achieve the necessary flexibility for rotation. As another example, the twistable element 45 having relatively movable parts could include a swivel, swivel-molded hook, bearings, or any assembly of parts having at least one degree of freedom that allows for reorientation of the fasteners 26.

During use, the wrist brace 10 is taken from a flat configuration (as shown in FIGS. 2-3 and 9-10) and applied to the hand, wrist and forearm of the wearer (as shown in FIGS. 1, 5, 6, 13, and 14). Application of the sheet of material 12 about the wrist of the wearer includes overlapping one of the lateral edges (e.g., lateral edge 21) over the other one of the lateral edges (e.g., lateral edge 20). In addition, the thumb tab 28 is extended between the thumb and index finger of the wearer.

Once the sheet of material 12 and thumb tab 28 have been at least partially applied to the wearer's anatomy, the fastening surfaces 34 on the outer surface of the fasteners 26 are pulled to the point of desired tension and secured to the complementary fastening surfaces 40 on the surface of the sheet of material. The optional splint 54 may also be inserted into the pocket 42 through the slit 11 if not already within the pocket for further support of the wearer's wrist.

Reversal of the wrist brace 10 to the opposite hand includes removal of the fasteners 26 from the surface of the sheet of material 12 and the sheet of material from the wearer's hand, wrist, and arm. Then, the splint 54 is removed from the pocket 42. Each of the fasteners is then reoriented by twisting or rotating the twistable elements 45 until the fastening surface 34 on its outer surface is coincident with the surface of the sheet of material 12 to be applied adjacent the wearer's hand, wrist, and arm. In this manner, when the lateral edges are overlapped, the fastening surfaces 34 can attach directly to the complementary fastening surfaces 40 on the newly reversed outer surface of the sheet of material 12. Advantageously, the fastening surfaces 34 of the fasteners 26 are located on only one surface so that the fastening surfaces are not free to snag on clothing or irritate the skin when attached to the sheet of material.

The present invention has many advantages. The construction of the twistable element 45 allows the wearer to quickly and easily rotate each of the fasteners 26 when switching between right and left-handed wrist brace configurations. Reversal of the fastening surface 34 when the fastening surface comprises at most one side of each of the fasteners ensures that the fastening surface is not exposed. This prevents abrasion of the wearer's skin by the fastening surface or snagging of the fastening surface on the wearer's clothing. Also, hiding or concealing the fastening surface 34 can improve the aesthetic appearance of the wrist brace. The use of multiple twistable elements 45 in conjunction with multiple fasteners (e.g., three twistable elements) at different positions along the lateral edge 21 of the sheet of flexible material allows adjustment of the amount of support provided by each of the fasteners at different locations.

Many modifications and other embodiments of the invention set forth herein will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. A reversible wrist brace for alternatively supporting both a right and a left wrist of a wearer, said reversible wrist brace comprising:
a sheet of flexible material having a first surface configured to overlie the right wrist of the wearer in a right-handed position, a second surface opposite the first surface configured to overlie the left wrist of the wearer in a left-handed position, a distal edge configured to extend at least partially around the wearer's hand, a proximal edge configured to extend at least partially around the wearer's forearm and a pair of opposing lateral edges capable of at least partially overlapping each other so as to extend around the wearer's wrist;
at least one fastener having a fastening surface and a non-fastening surface, said fastening surface configured to releasably attach to at least a portion of the first or second surfaces of the sheet of flexible material;
at least one connecting element having a first end connected to the sheet of flexible material, a second end connected to the fastener and a twistable element positioned between the ends, said twistable element configured to allow sufficient rotation between the ends so as to allow reorientation of the fastening surface between attachment to the second surface so as to secure the sheet of flexible material about the wearer's wrist in the right-handed position and attachment to the first surface so as to secure the sheet of flexible material about the wearer's wrist in the left-handed position.

2. A reversible wrist brace of claim 1, wherein said twistable element includes a first member attached to the first end and a second member attached to the second end and wherein one of said members defines an opening and the other one of said members is rotatably retained within said opening.

3. A reversible wrist brace of claim 2, wherein the second member is rotatable through 360° with respect to the first member.

4. A reversible wrist brace of claim 3, wherein the twistable element includes a third member extending between the first member and the first end, said third member attached to the first end and said first member rotatable with respect to the third member about an axis different than an axis of rotation of the first and second members.

5. A reversible wrist brace of claim 4, wherein the third member has a U-shape with a pair of arms and wherein the first member is rotatably supported between said arms.

6. A reversible wrist brace of claim 5, further comprising a strap extending through the U-shape between the U-shape and the first member, said strap attached to the sheet of flexible material at one end.

7. A reversible wrist brace of claim 1, wherein said twistable element includes a flexible tether.

8. A reversible wrist brace of claim 7, wherein the flexible tether is biased to an untwisted position and said fastening surface is coincident with one of the first and second surfaces of the flexible sheet of material.

9. A reversible wrist brace of claim 8, wherein the tether has a cross-section with a primary axis aligned with one of the first and second surfaces of the flexible material.

10. A reversible wrist brace of claim 1, wherein the twistable element is capable of at least 180° of twist.

11. A reversible wrist brace of claim 1, wherein the twistable element is capable of at least 360° of twist.

12. A method of reversing a wrist brace between right and left handed configurations, said method comprising:
extending a proximal edge of a sheet of flexible material of the wrist brace at least partially about a forearm, a distal edge of the sheet of flexible material at least partially about a hand and overlapping a pair of lateral edges of the sheet of flexible material so that the sheet of flexible material extends at least partially around one of a left and right wrists;
extending at least one fastener over an outer one of the lateral edges and attaching a fastening surface of the fastener to a first complementary surface on the sheet so as to secure the wrist brace about the one of the wrists;
detaching the fastening surface of the fastener from the first complementary surface of the sheet;
reversing the sheet and extending the sheet over the opposite wrist so that the first complementary surface is in contact with the opposite one of the wrists of the wearer;
twisting the fastener about a twistable element until the fastening surface is facing a second complementary surface on the sheet; and
extending the fastener over the outer one of the lateral edges and attaching the fastening surface of the fastener to the second complementary surface so that the wrist brace is secured about the opposite one of the wrists.

13. A method of claim 12, wherein twisting the fastening surface includes twisting the fastener about the twistable element at least 180 degrees.
